(19) United States
(12) Patent Application Publication
(10) Pub. No.: US 2005/0245995 A1
(43) Pub. Date: Nov. 3, 2005

(54) Transmitting and receiving device

(75) Inventor: Michael Diebold, Berlin (DE)

Correspondence Address: HAHN LOESER & PARKS, LLP, One GOJO Plaza, Suite 300, AKRON, OH, 44311-1076, US

(73) Assignee: BIOTRONIK GmbH & Co. KG

(21) Appl. No.: 11/115,034

(22) Filed: Apr. 26, 2005

(30) Foreign Application Priority Data
Apr. 30, 2004 (DE) 102004023654.2
Sep. 3, 2004 (DE) 102004043210.4

(51) Int. Cl. A61N 1/08

(52) U.S. Cl. 607/60

(57) ABSTRACT

Certain embodiments of the present invention disclose a data transmission unit for wireless communication with an electromedical implant and a data acquisition and evaluation center (home monitoring service center) wherein the data transmission unit is an electronic plug-in card which can be inserted into a base device, in particular into a mobile telephone, and which has at least a wireless data interface for the exchange of information with an electromedical implant, a wired data interface with the base device for the exchange of information between the plug-in card and the base device, at least one control unit for controlling the exchange of data by way of at least the wireless data interface, and a write/read data memory which is readable by the base device by way of the wired data interface, wherein the control unit is adapted to write data received from an external device by way of the wireless data interface into the write/read data memory.

### CROSS-REFERENCE TO RELATED APPLICATIONS/INCORPORATION BY REFERENCE

This application claims priority to German patent application serial number 10 2004 023 654.2 filed on Apr. 30, 2004, which is incorporated herein by reference in its entirety. This application also claims priority to German patent application serial number 10 2004 043 210.4 filed on Sep. 3, 2004, which is incorporated herein by reference in its entirety.

### TECHNICAL FIELD

Embodiments of the present invention relate to a data transmission unit. In particular, certain embodiments of the present invention concern a data transmission unit for a patient device for wireless communication with an electromedical implant and a data acquisition and evaluation center (e.g., a home monitoring service center).

### BACKGROUND OF THE INVENTION

Modern electromedical implants, in particular cardiac pacemakers, defibrillators and the like, offer a physician and patients a very high degree of security and comfort by means of so-called home monitoring functions.

In that situation, the implant records diagnosis and therapy information or technical data concerning the implant itself and transmits that information by way of a wireless data interface (telemetry interface) to an external portable patient device. From there the data are forwarded to the home monitoring service center by way of a second telemetry interface. In the home monitoring service center the data are stored and displayed for the physician. The physician can be informed directly about therapy progress and the current state of health of his patients. He thus has the option of reacting quickly to changes.

Without home monitoring, the physician can query those items of information only in the context of an examination of the patient. In critical situations, that can result in unwanted delays in the flow of information. In addition, any examination involves a considerable amount of time expenditure for the physician and the patient. Frequent examinations result in limitations in terms of mobility and quality of life in particular for the patient.

With home monitoring, the items of implant information are sent in the background by way of the patient device (see also U.S. Pat. No. 6,553,262 and U.S. Pat. No. 5,752,976) without the patient being limited in terms of leading a normal life. In other words, the patient has the security of monitoring by a physician without the stress of frequent examinations.

Indirect data transmission from the implant to the home monitoring service center by way of the portable patient device is effected in order to reduce the transmission power required on the part of the implant, the battery of which can only be changed, together with the entire implant, by a surgical intervention. Such a system and a method of doing that are set forth, for example, in U.S. Pat. No. 5,752,976.

The main aim and purpose of ongoing home monitoring represents the possibility of a rapid reaction to unexpected events so that the patient can be prevented from coming to harm. As the freedom of movement of a patient is not to be unnecessarily limited, data transmission to the home monitoring service center is preferably effected by way of a wireless communication network (GSM, UMTS, CDMA, WLAN and Bluetooth). Usually, for indirect data transmission, patient devices are constructed, in which a circuit of a mobile telephone and a circuit for implant communication are integrated in one device. That results in a patient device of relatively large dimensions which can only be used for medical-technical home monitoring of the patient or his electromedical implant.

Now, there is the problem that the patient always has to have about his person a relatively unwieldy device. Experience with looking after risk patients has shown that, in spite of its possibly life-saving significance, the device is frequently forgotten and ongoing home monitoring of the patient thus became an impossibility. If however the patient actually always has the device with him, that signifies that his quality of life is adversely affected just for the simple reason that the constant presence thereof continuously reminds him of his being at risk by virtue of the illness.

The high production costs of the patient devices which are produced in low numbers and which, in addition, have to be continuously electronically adapted to new mobile telephone circuits and standards are also a disadvantage. In addition, with a conventional patient device, the patient can only trigger pre-defined emergency situation actions as, in spite of the integrated mobile telephone, it cannot be used as such. If, for example, the patient would wish to have a consultation conversation with the physician, then in addition to the patient device he must also have a mobile telephone with him.

### SUMMARY OF THE INVENTION

In accordance with certain embodiments of the present invention, a data transmission unit is provided which permits communication between an electromedical implant and a home monitoring service center, but which in that respect signifies for the patient a lesser detrimental effect in respect of his quality of life and which is less likely to be forgotten so that medical home monitoring of the patient is less frequently interrupted. The data transmission unit should guarantee correct and secure transmission of the implant data, it should be inexpensive to produce and it should afford a high degree of flexibility in respect of use in current and future communication networks.

Such an embodiment of the present invention is attained by an electronic plug-in card which can be inserted into a base device and which includes at least a wireless data interface for the exchange of information with an electromedical implant, a wired data interface with the base device for the exchange of information between the plug-in card and the base device, at least one control unit for controlling the data exchange by way of at least the wireless interface and a write/read data memory which is readable by the base device by way of the wired data interface, wherein the control unit is adapted to write data received from an electromedical implant by way of the wireless data interface into the write/read data memory and to send data stored in the write/read data memory to an electromedical implant.

In most cases, the base device will be a mobile telephone. In the context of this application, however, mobile telephones are deemed to include all portable devices which permit data exchange by way of a mobile radio network or a wireless data network. So-called smartphones, Personal Digital Assistants (PDAs), etc. are also expressly embraced by the term even if speech communication is not provided at all or is provided only as a secondary function of the device.

From the point of view of the mobile telephone, the data transmission unit, according to certain embodiments of the present invention, functions like a memory card on which data from or for the implant are stored in given files. This ensures that a wide range of mobile telephones can detect and operate the data transmission unit without any problem and without additional I/O configuration or special driver software. In that way, the patient can expand any base device to provide a patient device for operation with his electromedical implant. No special technical knowledge is required for that purpose because it is only necessary for the data transmission unit, which is in the form of the electronic plug-in card, to be inserted into the mobile telephone.

Because of the far-reaching spread of mobile telephones, in that way a mobile telephone which the patient already has can be used for the purposes of medical home monitoring. Thus, the patient does not have to have an additional device with him and he is contactable at any time by the physician who is looking after him. In addition, the patient device which in the form of a mobile telephone with the data transmission unit according to certain embodiments the present invention, can also be used for normal telephone communication which, particularly in emergency situations, ensures quicker help by local rescue services. As in most cases, the patient is already accustomed to having a mobile telephone with him, and it becomes less likely that home monitoring will be interrupted because the patient has forgotten the device by mistake. The convenience aspect is also greatly enhanced for the patient as the electronic plug-in card scarcely occupies additional space and is integrated, almost without being noticed, into the mobile telephone. In addition, the manufacturing and development expenditure is reduced as the functionality of the mobile radio data interface does not have to be specifically implemented. It is additionally possible to save costs as possibly only one mobile telephone has to be operated. Home monitoring of patients can be effected easily by way of alternative or new wireless communication networks by the mobile telephone which accommodate the electronic plug-in card being replaced by a suitable device.

Updating of the software for operation of the data transmission unit in a mobile telephone or a change to the operating parameters of the electromedical implant can be easily effected in the presence of the physician looking after the patient, by way of a data connection from the mobile telephone. Hitherto, special programming devices were necessary for that purpose, by way of which the implant was configured by means of close-range telemetry. In that case, however, each model requires a programming device, which is matched thereto, from the manufacturer of the implant, as no standardized interface exists for the programming of electromedical implants. As however, just for reasons of space, a physician would like to be confronted only with a limited number of programming devices which in addition each involve their own respective idiosyncrasies in operation, he would have to limit himself to the use of models of individual manufacturers of electromedical implants, to the detriment of his patients.

It is important to protect the data which are transmitted between an implant and a home monitoring service center. In that respect, it is important to take account of a number of points of view. Besides general aspects such as data protection for the patient-related medical data, in particular, safety aspects also apply. As the home monitoring service center is required inter alia to perform the task of possibly triggering an alarm on the basis of the data received from the electromedical implant, it is indispensable for correct analysis of the medical condition of the patient that the data are correctly transmitted. Even more important, however, is the transmission of data to the patient device and the implant. If suitable operating parameters for a given patient, for example for a cardiac pacemaker, such as minimum and maximum heart beat rate, or the appropriate stimulation voltage, are ascertained by the home monitoring service center from the previously communicated medical data, or if an updated version of the operating software of the data transmission unit in a mobile telephone is to be transmitted thereto, correct transmission of those new operating parameters to the electromedical implant or the operating software to the mobile telephone should be ensured. If falsified operating parameters were to be used in the implant, the life of the patient in question would be put at risk. Defectively transmitted operating software could involve malfunctions in operation of a mobile telephone and a data transmission unit, to the stage of the mobile telephone being unusable. Therefore, the encoding methods known from the communications art should be used at every point in the transmission chain in order to be able to detect or correct transmission errors and achieve maximum certainty in data transmission.

In order, additionally, to ensure that the operating parameters or software originate from an authorized source, more specifically from the home monitoring service center, the transmitted data should also be protected by effective cryptographic encryption. For example, one or more private keys which are stored in the data transmission unit and which are protected from exchange can be used in that way to effectively prevent inadvertent or intentional re-programming of the electromedical implant or the mobile telephone. It will be noted that the expenditure for a specific decoding and decryption unit in the electromedical implant can be too high so that the data would have to be transmitted in non-encrypted form on the short-distance transmission between the data transmission unit and the electromedical implant. That can become inevitable, in particular, due to the indispensable low current consumption of the implant. In such a situation, under some circumstances, for security reasons or by virtue of applicable security requirements, it is desirable to provide only for reading out data from the implant, so that in no case can the implant be influenced from the exterior by the setting of altered operating parameters.

In accordance with an embodiment of the present invention, the write/read data memory of the electronic plug-in card is in the form of a non-volatile write/read data memory because, in that way, the data last transmitted and stored in the plug-in card are retained even after the operating voltage is switched off. It is possible, in that way, to prevent the loss of data because, for example, the battery of the mobile telephone is discharged.

Because, in accordance with certain embodiments of the present invention, the electronic plug-in card is to be capable of being inserted into a commercially available mobile telephone, it should comply, at least in regard to its dimensions and the wired interface to the mobile telephone, with the crucial standards, in particular the MMC, SD, SM, MemoryStick, and CF standards.

A variant of the data transmission unit has an encoding unit which encrypts medical or technical operating data of the electromedical implant prior to the transmission thereof to the home monitoring service center in order to guarantee the security of the transmitted data from unauthorized access and to prevent the replacement of the transmitted data by falsified data. In that way, the data protection requirements of some countries are also taken into consideration. The encoding unit can be implemented by suitable programming of the control unit of the card, which is in the form of a microcontroller.

The data transmission unit can also have a decoding unit which decrypts data which are transmitted in encrypted form from the home monitoring service center. That embodiment has the advantage that the data transmission from the home monitoring service center to the data transmission unit is also protected from unauthorized access. The decoding unit can also be implemented by suitable programming of the card control unit, which is in the form of a microcontroller.

In a variant of the last embodiment, the decoding unit is also adapted to check operating parameters for the electromedical implant, received from the home monitoring service center, for the correctness of such parameters, before they are transmitted to the implant. It is possible, in that way, to prevent defective programming of the implant, which would signify the health of the patient being put at risk.

In a similar manner, mis-programming of the mobile telephone, in the card slot of which the data transmission unit is operated, can be prevented insofar as, in an alternative configuration of certain embodiments of the present invention, the decoding unit is designed in such a way that it ensures the correct transmission of software for operation of the data transmission unit in the mobile telephone from the home monitoring service center to the patient device by checking the result upon decoding or decryption of the received operating software.

The wireless interface can communicate with one or more electromedical implants and other sensors. The frequency bands which are available for the wireless transmission of data are prescribed by international agreements and national regulations. The wireless data interface of the electronic plug-in card is, therefore, advantageously adapted to be able to effect data transmission at at least one such allowed frequency in accordance with the prevailing regulations. Particularly, it should be capable of effecting data transmissions in the frequency range of between 401 and 406 MHz.

It can advantageously be provided, in an embodiment of the present invention, that writing of the write/read data memory by way of the wired data interface is made impossible so that, instead of as normally for bidirectional communication between the implant and the home monitoring service center, the electronic plug-in card can only be used for the unidirectional communication emanating from the implant. In that way, it is possible to prevent the data sent from the electromedical implant being changed or overwritten prior to forwarding to the home monitoring service center, for example, by virtue of defective software of the mobile telephone or improper or unauthorized outside intervention and, thereby, a false alarm being possibly triggered by virtue of the altered data or indeed a genuine alarm being prevented.

In an alternative configuration, writing of the write/read data memory by way of the wired data interface is, in contrast, made possible in order also to be able to implement a communication of items of information (configuration parameters etc.) from the home monitoring service center to the electromedical implant.

In accordance with an embodiment of the present invention, the write/read data memory can be in the form of what is referred to as a dual-ported RAM, that is to say, a random access memory which can be simultaneously addressed by way of two different interfaces. In accordance with an embodiment of the present invention, the write/read data memory is linearly addressable.

In accordance with a certain embodiment of the present invention, the write/read data memory is connected between the wireless and wired data interface. Direct forwarding of the data from the wireless to the wired data interface and vice-versa is not possible in this embodiment.

As correct and complete communication of the data to, and possibly from, the home monitoring service center is important, an embodiment of the present invention provides a suitable energy store or buffer which can at least briefly maintain the current supply for the electronic plug-in card so that, as far as possible, a running transmission of data can still be terminated in the event of an abrupt interruption in the current supply by the mobile telephone.

In order to prevent the electronic plug-in card from being inadvertently removed from the mobile telephone while a data transmission is being conducted, a certain embodiment of the data transmission unit has a visual display which signals the implementation of a data transmission (link indicator). Such a display is also prescribed in many countries for reasons relating to data protection law or by regulations relating to radio data transmission and is also visible when the electronic plug-in card is inserted. Alternatively, the optical display can also be provided by way of the display of the mobile telephone by suitable programming of the operating software.

If the aim is that the electronic plug-in card can also be used by visually impaired persons, an alternative configuration can also have an acoustic signal generating device in order to acoustically signal the implementation of a data transmission. Alternatively, an acoustic signal generating device of the mobile telephone can be used, by means of suitable programming of the operating software.

In accordance with an embodiment of the present invention, the electronic plug-in card is of such a structure that it can be addressed and controlled on the part of the mobile telephone like a conventional data memory card. In that case, advantageously, there is no need for adapted or specifically programmed driver software for controlling the plug-in card. In such a configuration, the software of the mobile telephone uses the driver software for a usual data memory card in order to store data files on the data memory card or to read data files or to provide for writing access. The driver software then directly writingly or readingly accesses given memory regions of a data memory card and causes the desired data file operation by suitable control of the wired data interface. As the electronic plug-in card, according to certain embodiments of the present invention, shows itself as a normal data memory card, at least certain actions in respect of the electronic plug-in card can be triggered and controlled on the part of the mobile telephone by writing or reading access to data files on the assumed data memory card, without adaptation of the operating system of the mobile telephone having to be effected, for example, by special driver software. In that respect, it is irrelevant whether memory of the nature and size appropriate to the usual driver software for data memory cards is actually present on the electronic plug-in card.

If reading from and writing to the write/read data memory by the two data interfaces coincide in time, that can entail falsification of the data which are read out as under some circumstances they are then composed of data which, in part, were already present prior to the current writing access while, for another part, they were already changed by the writing access being implemented. Certain embodiments of the present invention, therefore, provide a control unit which is adapted to prevent simultaneous or time-overlapping reading-out and writing, or to co-ordinate such operations in such a way that the data which are read out either correspond to those which were present in the data memory prior to the writing access or, however, those which are just being written into the data memory. Alternatively, successive writing accesses could be effected to different, non-overlapping addresses of the write/read data memory so that the data of two successive writing accesses can never mutually overwrite.

An alternative embodiment of the present invention has a second write/read data memory which provides a copy of the data contained in a part of the first write/read data memory, which is to be overwritten by a writing access being conducted, for a simultaneous reading access.

If a patient feels unwell, he should be in a position to trigger an immediate communication of current medical data to the home monitoring service center. For that purpose, a certain embodiment of the present invention provides an operating element such as, for example, a button by which communication of data can be triggered.

In accordance with an embodiment of the present invention, the electronic plug-in card has a ROM (read only memory), or flash ROM, which contains program instructions and data for execution by a control unit of the electronic plug-in card. In accordance with a variant embodiment of the present invention, the ROM or flash ROM contains, in addition or instead, program instructions and data for execution by the mobile telephone. As a result, a program, for example, for controlling the communication with the implant is immediately available after switching on the operating voltage and can be loaded into the mobile telephone or can pass into the electronic plug-in card itself for execution by a control unit.

A further variant embodiment of the electronic plug-in card, according to the present invention, has a RAM which receives data produced in the execution of program instructions. The data can be produced, on the one hand, when program instructions are executed by the control unit or, on the other hand, by the mobile telephone.

### BRIEF DESCRIPTION OF SEVERAL VIEWS OF THE DRAWINGS

The invention is described in greater detail hereinafter by means of an embodiment with reference to the Figures.

FIG. 1 provides an overview of the electronic components of an electronic plug-in card, in accordance with an embodiment of the present invention, and the mobile telephone as the base device for operation of the plug-in card.

FIG. 2 shows a block diagram of the software components of the plug-in card and the mobile telephone, in accordance with an embodiment of the present invention.

FIG. 3 shows a plan view of an implementation of the data transmission unit, in accordance with an embodiment of the present invention, wherein the dimensions satisfy the respectively prevailing standards of multi-function cards (SD, MMC, etc.).

FIG. 4 shows a side view of the electronic plug-in card, in accordance with an embodiment of the present invention.

### DETAILED DESCRIPTION OF THE INVENTION

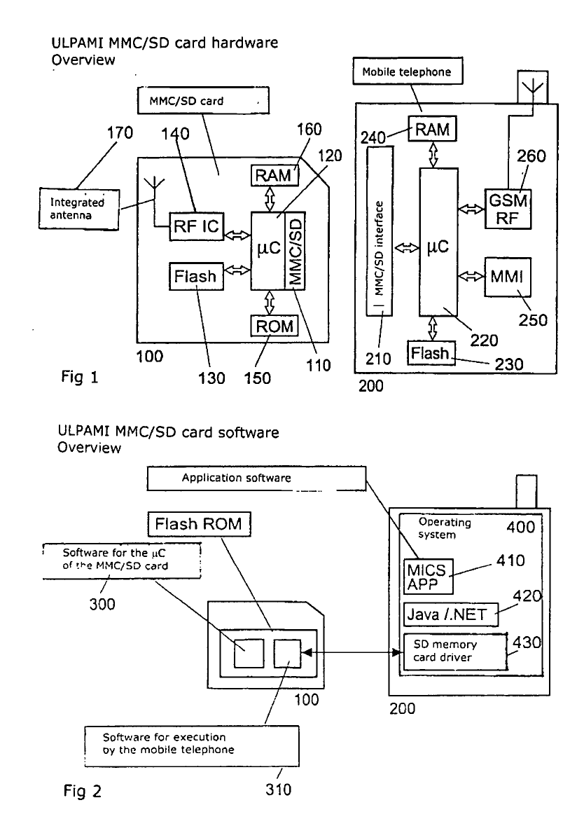

FIG. 1 shows block diagrams of an electronic plug-in card 100 in accordance with the MMC and SD standards (at the left) and a mobile telephone 200 (at the right). The dimensions of MMC and SD cards are similar or identical so that many mobile telephones can accommodate MMC and SD cards by way of the same slot. The MMC/SD card 100 includes an antenna 170, a RAM 160, a ROM 150, a wireless data interface component 140, a write/read data memory 130 in the form of a non-volatile flash memory, a control unit 120 and a wired interface 110, which are illustrated in FIG. 1 by seven corresponding blocks. The wireless data interface component 140 includes the components required for communication with electromedical implants. The block identified by “μC” includes a programmable control unit 120 which is in the form of a microcontroller and which performs various control tasks on the MMC/SD card. Thus, it controls, in particular also, data exchange by way of the wired interface 110 identified as “MMC/SD” with the mobile telephone 200, or provides for encryption and decryption of the implant data.

In the embodiment of the MMC/SD card illustrated in the block diagram, the microcontroller 120 is arranged centrally and communicates with the other blocks by way of bidirectional buses which are illustrated as arrows. As microcontrollers essentially represent programmable control units, this structure makes it possible with just one central component to control and execute both writing and reading of the write/read data memory 130, and also communication by way of the wireless data interface 140. In that case, the ROM 150 and the RAM 160 stand beside the microcontroller 120 and contain the program code for the microcontroller 120 or receive data produced in execution thereof. Instead of a fixed-wired ROM, it is also possible to provide another non-volatile memory, thus for example, a flash ROM. Current microcontrollers often include RAM and a non-volatile memory for accommodating program code so that the system structure is simplified. If such a microcontroller is used on the electronic plug-in card 100, the illustrated ROM and RAM blocks 150 and 160 are not implemented separately.

The second block diagram shows some central components of a mobile telephone 200. It serves only to illustrate a typical device which is to accommodate an electronic plug-in card 100.

The mobile telephone 200 also has a control unit 220 in the form of a microcontroller (μC), flash memory 230, RAM 240, and a wireless GSM data interface 260 with antenna. In addition, a wired data interface 210 to the MMC/SD card is illustrated at the left by the block “MMC/SD interface”. A functional unit which is thus or similarly not also present in the MMC/SD card is a “man-machine interface” 250 (MMI), that is to say, a user interface with display and keypad. The wireless GSM data interface 260, which is referred to as the “GSM RF”, permits wireless communication between the mobile telephone 200 and a GSM base station.

FIG. 2 shows a similar diagram to FIG. 1. It again diagrammatically shows the MMC/SD card 100 and a mobile telephone 200. In this case, however, the blocks represent the software components which are required for operation. On the electronic plug-in card 100, the flash ROM 130 includes, on the one hand, software for the microcontroller 120 of the plug-in card 100 (300), and on the other hand, the software which is to be executed in the mobile telephone (310). That software 310 is loaded from the plug-in card 100 to the mobile telephone 200 and there executed by the μC 220.

The software components of the mobile telephone 200 are illustrated arranged hierarchically from above downwardly. In front of the background of an operating system 400 of the mobile telephone 200, identified here as the “phone OS”, which controls all the software to be executed as well as the hardware, that is at the very top the application software 410 for the operation of the MMC/SD card and a possible operating surface for the patient (“MICS APP”). Illustrated therebeneath is a JAVA or “NET” interpreter 420 which executes the program code of the application software written in one of the two languages, by interpretation in relation to running time (“Java/.NET”). Which language is used depends, in that respect, on the types of mobile telephone employed. Alternatively, it would also be possible to use dedicated programs for a given mobile telephone, which are executed not by an interpreted but directly by the mobile telephone.

Disposed logically lowermost is the driver software 430 for an SD memory card and is suitably entered under the other blocks, by means of which the operating system 400 of the mobile telephone 200 communicates with the electronic plug-in card 100 (“SD memory card driver”). That driver software 430 is contained as standard in all mobile telephones which permit an MMC/SD memory card to be accommodated and operated. As the electronic plug-in card 100, on the part of the mobile telephone 200, behaves like a normal memory card, the same driver software 430 can be used for operation of the plug-in card 100, as for conventional memory cards. As a result, new driver software does not have to be specifically programmed for each type of mobile telephone which is possibly used, and that represents a great advantage in carrying out certain embodiments of the present invention.

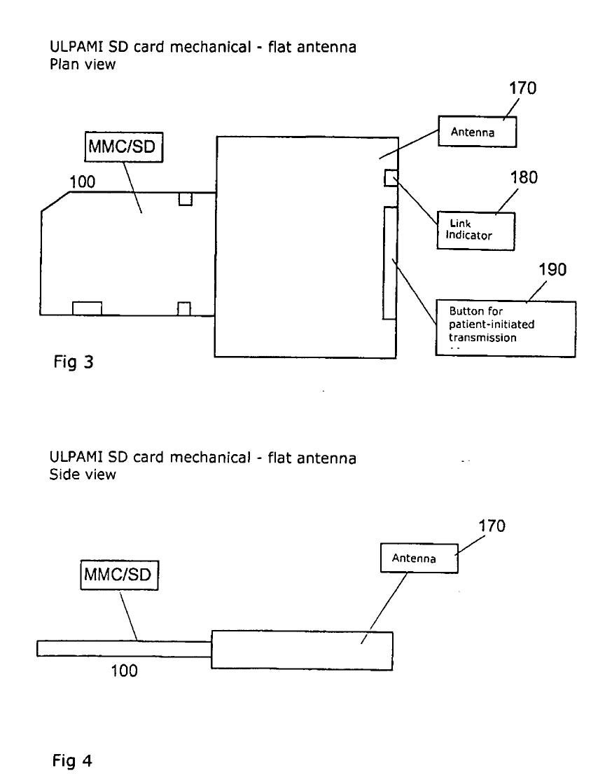

FIG. 3 shows a view of an electronic plug-in card 100 in accordance with the SD standard. The plug-in card 100 is divided into a part which is shown at the left and which can be inserted into the mobile telephone 200, and a somewhat larger part which projects out of the device, on the right-hand side of the illustration. The part shown at the left is in accordance with the specifications relating to the dimensions of an SD card. The part shown at the right has a red light emitting diode which is illustrated at top right and which forms a link indicator 180 which signals the various operating states of the SD card but, in particular, a data communication which is being implemented between the electromedical implant and the SD card 100. Also provided at the end of the plug-in card 100, which is illustrated at the right, is a button 190, the actuation of which triggers transmission of data to the home monitoring service center. The drawing does not show contacts of the wired data interface 110, which are disposed at the left-hand end on the underside of the plug-in card 100. The illustrated variant has a flat antenna 170 which is integrated into the part that protrudes from the mobile telephone.

FIG. 4 shows a side view of the data transmission unit 100 in the form of an SD plug-in card. Once again, shown at the side on the left, is the thin part which can be inserted into the mobile telephone while, shown at the right, is the part which protrudes from the mobile telephone.

1. A data transmission unit for wireless communication with an electromedical implant and a data acquisition and evaluation center, said data transmission unit comprising an electronic plug-in card which can be inserted into a base device, said electronic plug-in card including at least:
a wireless data interface for the exchange of information with an electromedical implant;
a wired data interface with the base device for the exchange of information between the plug-in card and the base device;
at least one control unit for controlling the exchange of data by way of at least the wireless data interface; and
a write/read data memory which is readable by the base device by way of the wired data interface, and wherein the control unit is adapted to write data received from an external device by way of the wireless data interface into the write/read data memory.

2. The data transmission unit as set forth in claim 1, wherein the write/read data memory, being readable by the base device by way of the wired data interface, is in the form of a non-volatile write/read data memory.

3. The data transmission unit as set forth in claim 2, wherein the electronic plug-in card complies with the requirements of a MMC standard (MultiMedia Card), a SD standard (Secure Digital), a SM standard (SmartMedia), a MemoryStick standard, or a CF standard (Compact Flash).

4. The data transmission unit as set forth in claim 3, wherein the data transmission unit includes an encoding unit which is directly or indirectly connected to the wired and wireless data interfaces and is adapted to encrypt medical data acquired by an electromedical implant by way of the wireless data interface or technical data concerning the electromedical implant itself in such a way that the encrypted data can be read out by way of the wired data interface.

5. The data transmission unit as set forth in claim 4, wherein the data transmission unit includes a decoding unit which is directly or indirectly connected to the wired data interface and is adapted to decrypt data received by way of the wired data interface.

6. The data transmission unit as set forth in claim 5, wherein the decoding unit is directly or indirectly connected to the wireless data interface and is adapted to check encoded or encrypted parameters received by way of the wired data interface for the operation of an electromedical implant prior to the transmission thereof by way of the wireless data interface to said implant for the integrity of the parameters and the correct transmission thereof to the data transmission unit.

7. The data transmission unit as set forth in claim 5, wherein the decoding unit is adapted to check encoded or encrypted operating software received by way of the wired data interface for operation of the data transmission unit in a base device for the integrity of the operating software and the correct transmission thereof to the data transmission unit.

8. The data transmission unit as set forth in claim 6, wherein the wireless interface is adapted to transmit data at a carrier frequency of between 401 and 406 MHz.

9. The data transmission unit as set forth in claim 8, wherein the write/read data memory is not writable by way of the wired data interface.

10. The data transmission unit as set forth in claim 8, wherein the write/read data memory is writable by way of the wired data interface.

11. The data transmission unit as set forth in claim 10, wherein the write/read data memory is in the form of a dual-ported RAM.

12. The data transmission unit as set forth in claim 11, wherein the write/read data memory is connected between the wireless and the wired data interfaces.

13. The data transmission unit as set forth in claim 12, wherein the electronic plug-in card includes a suitable energy storage means or buffer which briefly supplies the electronic components of the plug-in card with current if the current supply for the plug-in card by the base device is interrupted.

14. The data transmission unit as set forth in claim 13, wherein the electronic plug-in card has an optical display which is visible when the electronic plug-in card is inserted into a mobile telephone and is adapted to deliver a signal when a data exchange takes place by way of the wireless data interface.

15. The data transmission unit as set forth in claim 14, wherein the electronic plug-in card has an acoustic signal generating device which is adapted to deliver an audible signal when a data exchange takes place by way of the wireless data interface.

16. The data transmission unit as set forth in claim 15, wherein the wired data interface is so designed that the electronic plug-in card can be controlled and used like a standard data memory card.

17. The data transmission unit as set forth in claim 16, wherein the control unit of the electronic plug-in card is adapted to prevent simultaneous reading-out from and writing to the write/read data memory or to coordinate simultaneous writing and reading accesses in such a way that the read-out data are in their entirety either identical to those data which were present in the write/read data memory prior to the beginning of the writing access or identical to those which are present in the write/read data memory after the end of the writing access.

18. The data transmission unit as set forth in claim 17, wherein the electronic plug-in card additionally includes a second write/read data memory which is adapted to provide a copy of the information contained in the first write/read data memory prior to a writing access which is being carried out to the first write/read data memory for simultaneous reading accesses by way of the respective other data interface.

19. The data transmission unit as set forth in claim 18, wherein the electronic plug-in card has an operating element which can be operated when the plug-in card is inserted and by which data transmission by way of one of the data interfaces can be triggered.

20. The data transmission unit as set forth in claim 19, wherein the electronic plug-in card includes a ROM or a flash ROM with program instructions and data for execution by the control unit.

21. The data transmission unit as set forth in claim 20, wherein the electronic plug-in card includes a ROM or a flash ROM with program instructions and data for execution by the base device.

22. The data transmission unit as set forth in claim 21, wherein the electronic plug-in card has a RAM for the intermediate storage of data which are produced upon execution of program instructions.

23. The data transmission unit as set forth in claim 1, wherein the base device comprises a mobile telephone.

24. The data transmission unit as set forth in claim 1, wherein the data acquisition and evaluation center includes a home monitoring service center.

25. The data transmission unit as set forth in claim 7, wherein the wireless interface is adapted to transmit data at a carrier frequency of between 401 and 406 MHz.

26. The data transmission unit as set forth in claim 25, wherein the write/read data memory is not writable by way of the wired data interface.

27. The data transmission unit as set forth in claim 25, wherein the write/read data memory is writable by way of the wired data interface.
